(19) United States
(12) Patent Application Publication
(10) Pub. No.: US 2023/0033381 A1
(43) Pub. Date: Feb. 2, 2023

(54) NANOFIBROUS WOUND DRESSING

(71) Applicants: BURSA ULUDAG ÜNIVERSITESI, Nilüfer, Bursa (TR); INOVENSO TEKNOLOJI LIMITED SIRKETI, Istanbul (TR)

(72) Inventors: Esra KARACA, Bursa (TR); Cansu ARAS, Bursa (TR); Elif Tümay ÖZER, Bursa (TR)

(21) Appl. No.: 17/784,503

(22) PCT Filed: Dec. 31, 2019
(86) PCT No.: PCT/TR2019/051266

§ 371 Date: Jun. 10, 2022

(51) Int. Cl. A61L 15/22; D01D 5/00; D01F 1/10; D01D 1/02; D04H 3/03; D01F 6/70; D04H 3/009

(52) U.S. Cl. CPC A61L 15/225; D01D 5/0038; D01F 1/10; D01D 1/02; D04H 3/03; D01F 6/70; D04H 3/009; A61L 2400/12; A61L 2300/30; D10B 2509/022; D10B 2331/10

(57) ABSTRACT

The invention is related to the nanofibrous wound dressing that has been developed to be used in the treatment of various types of skin wounds including chronic and acute wounds in the biomedical sector, wherein said wound dressing comprises a bioactive agent that promotes the process of wound healing.

### TECHNICAL FIELD

The present invention relates to nanofibrous wound dressing that has been developed to be used in the treatment for a variety of skin wounds including chronic and acute wounds in the biomedical sector, wherein said nanofibrous wound dressing comprises a bioactive agent that has beneficial effect on the process of wound healing.

The invention is in particular related to nanofibrous wound dressing provided to be used in the treatment of a variety of skin wounds including chronic and acute wounds in the biomedical sector, wherein said nanofibrous wound dressing has been produced by the use of the method of electrospinning from a polyurethane polymer solution containing black seed oil (nigella sativa) as an additive.

### PRIOR ART

The disease is defined as a wound that has occurred as a result of the disintegration of epithelial tissue after a trauma or surgical operation. Different types of wounds are already known depending on the way they occur. These types of wounds can be categorized as chronic wounds that result from some diseases such as ulcers, diabetes and recurring wounds that recur at certain periods of time as well as acute wounds that occur as a result of deep tissue injuries, a surgical laceration or sudden physical trauma.

Wound dressings are materials that are used for covering wounds that are used to substitute the natural epithelium that has been lost as a result of an injury. The process of wound healing is based on the use of suitable wound dressings as well as on the medication to be administered. As the process of wound healing comprises various phases, different wound dressings may be required to be used in every phase. A wound dressing has the functions such as covering the wound area, protecting the wound from trauma, bacterial invasion, and hazardous materials, preventing the wound from drying by allowing the liquid that has formed at the wound area to evaporate, stopping bleeding, and providing the most suitable cellular activity and the requirements for rapid healing for the wound. Therefore, the wound dressings being used in the process of wound treatment and healing are of great importance.

The features of the wound dressing to be used can also vary depending on the type of wound and the requirements of the process of wound treatment. Accordingly, a great variety of wound dressings have already been provided according to their raw materials, physical forms, and the active ingredients they contain. The most frequently used wound dressings have been summarized in Table 1.

TABLE 1
The most frequently used wound dressings
and the usage areas thereof

| Wound | |
|---|---|
| Dressing | Usage Area |
| Absorbance | Absorption of hard exudates |
| Alginate | Autolytic debridement, hard exudate |
| | drainage, infection method |
| Composite | Wound treatment combination |
| Foam | Autolytic debridement, hard exudate |
| | drainage, infection method |
| Hydrocolloids | High moisture vapor transmission, |
| | autolytic debridement, discharge |
| | of a slight flow of fluid |
| Hydrogels | Autolytic debridement, slight/moderate |
| | exudate drainage, infection method |
| Collagenase | Enzymatic debridement |
| Silicone | Reducing of hypertrophic and keloid scars |
| Wound fillers | Protecting moist medium, exudate |
| | method |
| Silver | Infection method, antibacterial activity |

Improved wound dressings including foam, adhesive films, hydrogels, alginates, hydrocolloids, and biological dressings have already been used frequently in the prior art. However, said wound dressings are unable to fully provide some requirements such as gas and water vapor exchange, the ability to absorb liquid that would contribute to rapid wound healing. Many studies assert that the additives that ensure an antibacterial aspect for the wound dressing such as silver have some toxic effects. However, today, in modern approaches of wound care, an ideal wound dressing is anticipated to have a porous structure for water and gas permeability, to be able to protect the wound against the risk of an infection, to be biocompatible prevent any toxic effects, to have a certain level of adhesion, and to be designed to enable cell growth, which all constitute the features of natural skin. In this context, the macrostructures that are produced by the use of conventional production methods are unable to provide similar features to the morphological features of the natural skin, and thus novel developments as to wound dressings have been needed in the related technical field.

In the prior art, the studies on nanofibrous wound dressing that are extruded with the use of electrospinning have also been provided in the technical field. But, the additives that promote the process of wound healing are mostly composed of synthetic or metallic based additives. In most of the studies carried out, it has been asserted that these additives would create some toxic effects inside the body if they are used in wound dressings. Limited availability and high costs of said additives bring about another critical problem. A product that has been obtained by this means leads to disadvantages concerning both its compatibility with the body and raw materials and cost factors in its manufacturability.

In the patent and literature search conducted as to the prior art, an international application document with the publication number of WO2010097799 A1 has been discovered. In said patent application document, a biocompatible, porous wound dressing is disclosed wherein said wound dressing is produced with the method of electrospinning from polyurethane-silicone copolymer, polyurethane carbonate, polycarbonate polymer group and combinations of said polymer.

An American patent application with the publication number U.S. Pat. No. 8,728,498 B2 pertains to nanofibrous wound dressing that is obtained from the mixture of silk fibroin/polyethylene oxide polymer by being drawn with the use of the electrospinning method.

Another US patent application with the publication number of U.S. Pat. No. 9,101,508B2 describes a wound dressing that is produced with the method of electrospinning from the chitosan polymer containing the vegetable extract of Melilotus officinalis (yellow sweet clover).

A European patent application with the publication number of EP3500313 A1 discloses a wound dressing that is produced with the method of electrospinning from polycaprolactone (PCL) polymer.

A Korean patent application numbered KR101628205 B1 is related to a wound dressing that is produced with the method of electrospinning from polylactic acid and polycaprolactone polymers and various combinations thereof.

Another Korean patent application with the publication number of KR20190023871 A discloses a wound dressing that is produced with the method of electrospinning from the polyurethane polymer mixture containing silver nanoparticles.

A further Korean patent application numbered KR20180000884 A is related to a wound dressing that is produced with the method of electrospinning from a gelatin/polyurethane polymeric mixture.

In conclusion, as mentioned above, many problems and disadvantages have been encountered in the related technical field, and current applications fail to solve these problems and drawbacks. This requires providing development and novelty for the technical area.

### BRIEF DESCRIPTION OF THE INVENTION

The present invention is related to nanofibrous wound dressing that has a beneficial effect on wound healing, which meets all of the aforementioned requirements, eliminates all of the aforementioned disadvantages and provides some additional advantages to the field of art.

The main aim of the invention is to obtain nanofibrous wound dressing to be used in the treatment of the skin wounds, wherein said wound dressing is produced by means of the electrospinning method, from polyurethane polymer containing black seed oil. The active components such as thymoquinone in particular that are present at the essential oil part of the black seed oil contained in the content of the nanofibrous wound dressing subject to the invention have several wound-healing effects including antimicrobial, anti-inflammatory and antioxidant. Furthermore, the black seed oil has a high potential of antioxidant activities and thus it helps to regulate the enzymes and catalyzers that have an active role in the process of wound healing and promotes the free radicals to be maintained at a certain level. Therefore, the black seed oil contained in the nanofibrous wound dressing drawn by electrospinning inhibits oxidative stress and exhibits antioxidant activity.

Another aim of the invention is to provide a wound dressing that is capable of promoting wound healing. Nanofibrous mat that has been obtained with the invention ensures oxygen and air permeability by means of a wide surface area and the nanoporous structure it has, while it acts as a barrier against bacteria.

A further aim of the invention is to provide a wound dressing that exhibits similar features with morphological features of the extracellular matrix of the skin including hemostatic features due to the method of electrospinning, humidity absorption, and breathing capability. In the scope of the invention, very small diameters of fiber can be attained on the nanofibrous mat obtained by the use of the electrospinning method and accordingly, a very suitable environment for the development of cells and the formation of three-dimensional cellular colonies can be obtained. This case promotes the formation process of new cellular structures.

In an attempt to achieve all of the aforementioned aims and to provide a solution to the problems known from the prior art, the invention is related to nanofibrous wound dressing; said wound dressing comprising black seed oil and polymer.

The invention is a production method of a wound dressing in an effort to achieve all of the aforementioned aims and to provide a solution to the problems known from the prior art, said production method comprising the following steps of the process;
extracting black seed oil from black seed,
dissolving the polymer in a solvent,
adding the black seed oil into the polymer solution obtained,
obtaining nanofibrous mat with the method of electrospinning from the solution containing the polymer, solvent and black seed oil.

Structural and characteristic features and all of the advantages of the invention will be understood more clearly with the help of the detailed description and drawings presented below. Therefore, any evaluations as to the invention should be done considering said detailed description and drawings.

### DRAWINGS OF THE INVENTION

FIG. 1 shows a general perspective view of an electrospinning unit through which the wound dressing subject to the invention has been produced.

FIG. 2 illustrates the molecular structure of the thermoplastic polyurethane.

FIG. 3 shows SEM images of nanofibrous mats

FIG. 3a shows an image of Polyurethane;

FIG. 3b; shows images of the Polyurethane containing black seed oil.

### DETAILED DESCRIPTION OF THE INVENTION

In this detailed description, preferred embodiments of the invention have been disclosed for a better understanding of the subject matter and without limitation.

The invention is related to nanofibrous wound dressing to be used in the biomedical sector. The nanofibrous mat that forms said wound dressing contains black seed oil and polymer. As polymer, those to be selected out of polyurethane (PU) polymer, polyacrylonitrile (PAN) polymer, polyester (PES) polymer, polylactic acid (PLA), cellulose acetate (CA), poly (ε-caprolactone) (PCL), polyethylene oxide (PEO), alginate, polyvinyl alcohol, fibroin, hyaluronic acid, polycaprolactone, and similar polymers are used. In the invention, the preferred polymer; is polyurethane (PU) polymer. Throughout the description, the term “nanofiber” refers to the fibers having a diameter of less than one micron.

The wound dressing subject to the invention contains polymer in the range of 5% -18% by weight, black seed oil in the range of 5% -20% by volume, and a solvent in the range of 75% -95% by volume. In a preferred embodiment of the invention, said wound dressing contains 10% polymer by weight, 10% black seed oil by volume, 80% solvent by volume.

Polyurethane is used as a polymer in the composition according to the invention. Said polyurethane; is a polymer that is the final product of a reaction of a polyol (alcohol containing more than two reactive hydroxyl groups per mole (—OH) with a di or poly-isocyanate (—N═C═O) that occurs in the presence of a suitable catalyzer, a chain extender, and inactive ingredients. Organic units on the polymer chain are joined to each other by the urethane bonds that repeat between themselves (—NH—CO—O—). The polyurethane structure comprises a polyether or polyester-based soft segment and an isocyanate based hard segment. The hard segment disperses in the soft segment and molecules are linked to each other through the urethane bonds. Polyurethanes can be synthesized under thermoplastic or thermoset conditions. The polyurethanes having a linear macromolecule chain structure that exhibits similar behavior with an elastomeric material are called thermoplastic polyurethanes (TPU). In FIG. 2, the molecular structure of the thermoplastic polyurethane is illustrated. With the use of thermoplastic polyurethane (TPU) polymer, suitable solvents for electrospinning can be prepared.

Black seed oil has been used as an additive in polyurethane. It is obtained from Nigella sativa (NS) species of Ranunculaceae (The Buttercup Family) and it has a long history of use as a therapeutic plant since ancient times.

The black seed plant has high nutritional values and contains a variety of active components. The primary components making up the structure of the seed are; 0.4-0.45% essential oils, 31-35.5% saturated/unsaturated fixed oils, 33-34% carbohydrates, 16-19.9% proteins, 4.5-6.5% fiber, 3.7-7% ash (calcium salts), 0.013% saponins, 5-7% moisture.

One of the vegetative resources that have been traditionally utilized in the treatment of wounds and burns is the black seed and extracts thereof. The bioactive components contained in the fixed/essential oil of the black seed oil have therapeutically and pharmacologically important features. A great number of studies have been provided to show that wound healing aspect and the other therapeutic features of black seed and its extracts including antioxidant, antihistamine, anti-inflammatory, antimicrobial, anticancer are a result of the bioactive components they contain, and particularly, of the thymoquinone which is a major bioactive component of the essential oil.

In the production method of the wound dressing subject to the invention, first of all the seed oil is extracted. Said black seed oil is extracted from the black seed (Nigella sativa) through the use of the method of cold pressing. In this step of the process, a cold pressing machine is used, and the operating temperature of said machine is preferably 40° C. After completing the step of the invention in which the black seed oil is extracted, the polymer is stirred into the solvent until it has a concentration rate in the range of 5% -18% (w/v) by weight. As polymer, those to be selected out of polyurethane (PU) polymer, polyacrylonitrile (PAN) polymer, polyester (PES) polymer, polylactic acid (PLA), cellulose acetate (CA), poly (ε-caprolactone) (PCL), polyethylene oxide (PEO), alginate, polyvinyl alcohol, fibroin, hyaluronic acid, polycaprolactone, and similar polymers are used. In the invention, the polymer is able to be dissolved in the solvent systems that have been prepared by mixing one or more than one of the solvents including dimethylformamide (DMF), dimethyl sulfoxide (DMSO), tetrahydrofuran (THF), chloroform, dichloromethane at certain ratios.

The black seed oil in the range of 5% to 20% (v/v) (preferably 10% (v/v)) by volume of the total mixture is added into the mixture obtained after the polymer is dissolved in the solvent. In preparation of the mixture, polymer chips are added onto a certain amount of solvent contained in an inert glass vial and are continuously stirred in a magnetic mixer at the temperature range of 30° C. to 45° C. (preferably 45° C.), and hence said polymer chips are enabled to be dissolved. Subsequently, the black seed oil additive is added into this solution obtained, and the solution is completed to any desired volume by adding solvent. Said solution is preferably stirred in the magnetic mixer for 24 hours at the room temperature and hence a homogenous solution is obtained.

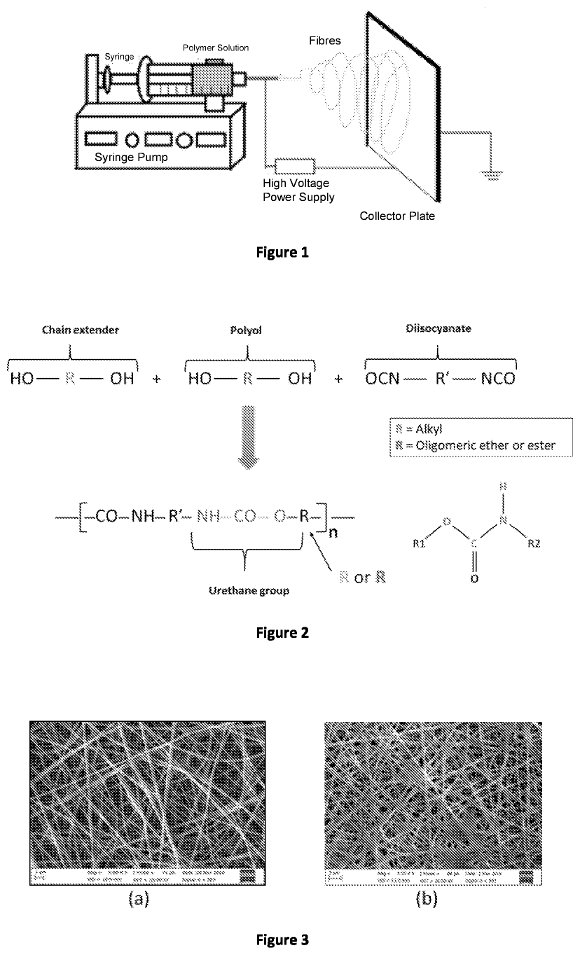

The next step of the process is the process of electrospinning. The method of electrospinning is more commonly preferred in the production of nanofibers due to its multifunctionality, operational compatibility with most of the polymers, and suitability for the production of proper nanofibers at low cost compared to other methods for the production of nanofibers. The basic principle of the method of electrospinning is constituted of obtaining nanofibers from a molten or dissolved polymer by the effect of electric field strengths. An electrospinning system is basically composed of; a power source, a feeding unit and a neutral collector (grounded) plate in which the nanofibers produced are accumulated. In the process of electrospinning; the dissolved or molten polymer is continuously fed to a nozzle of a syringe pump, by means of the usage of a controllable pump in the feeding unit. Surface tensions affect the polymer solution at the nozzle of the syringe pump. With the effect of the surface tensions, the polymer solution in liquid state is suspended in a droplet state at the nozzle of the syringe pump. The electrical strengths that have been formed by the voltage (V) applied by the power source act by pushing the surface tensions that act on a surface of the drop. Due to an increase in the electrical strengths acting on the drop suspended which occurs as a result of an increase in the voltage rate applied, and the viscoelasticity of the polymer, stretching occurs in the drop to an extent. When the voltage applied reaches a critical value (VC); it overcomes surface tension forces of the electrical strengths that occur on the polymer surface and afterward, the drop takes a shape called Taylor cone. When the voltage applied exceeds the critical value (V>Vc); a polymer jet extends out of an end of the Taylor cone and moves from the electrical area to the neutral collector plate. Repelling forces of the polymer jet created, has an axial component that extends said jet toward the collector. According to the flow rate measurements carried out, as it moves away from the Taylor cone, the variance of the jet rate increases as well as the jet rate. In this regard, the jet extending out of the Taylor cone steadily moves for a certain period of time while it gradually increases its speed. As a result of this case, the diameter of the jet swiftly diminishes when the jet starts to extend and the solvent starts to evaporate. As the jet tapers off, the surface load of the jet per unit area decreases while the surface load of said jet per unit mass increases. As the polymer proceeds steadily and properly, the charges that act on the jet surface mutually propel each other and they are separated from the jet, and thus whipping instability occurs. Said polymer that exhibits whipping instability separates from its jet and proceeds to the neutral collector plate after being divided into very fine fibers. During the travelling of the fibers inside the electrical area formed between the feeding unit and the collector plate, said fibers dry and harden due to the evaporation of the solvent, and ultimately the reticular nanofibers accumulate on the neutral plate. A general view of the electrospinning unit through which the wound dressing subject to the invention is produced is represented in FIG. 1.

In the method according to the invention, the solution that has been prepared in the previous step of the process and that has been stirred until it becomes homogenous is subjected to electrospinning such that the feeding rate is preferably in the range of 0.5 mL/hour-1.5 mL/hour (preferably 0.7 mL/hour), the distance between the feeding unit and the collector is preferably 8 cm to 30 cm (preferably 20 cm) and the voltage rate applied is in the range of 6 kV-30 kV (preferably 15 kV). The production of the nanofibrous mat is performed at room temperatures.

Furthermore, the nanofibrous mat can also be produced under the same conditions from the polymer solution that does not contain black seed oil. SEM images of two different surfaces obtained are represented in FIG. 3.

To comparatively examine the activity of the wound dressing according to the invention, the wound size decrement occurring on the wound surface of the wound dressings that have been prepared with the use of the method disclosed in the detailed description mentioned above are measured on day 0, 1, 3, 5, 7, 9 and 21 with the planimetric method.

This experiment that has been performed with a control group is herein presented only as an example and without limitation. To perform the comparison mentioned, a transparent acetate film is placed on the wound and the boundaries where the wound area intersects with intact tissue are pinpointed with an acetate pencil. The wound forms obtained are scanned and transferred to a computer and then they are defined as “cm²” in the ImageJ software, and the percentage of the wound size decrement is calculated through the use of the formula given below.

Wound Size Decrement(%)=100−[A_{wound(n)}/A₀]*100

Wherein; A₀: The initial wound area (Day 0) and A_{wound(n):} n. The wound area on the day of measurement.

In addition to this, the performance of the wound dressing subject to the invention in in vivo environment has been assessed by comparison with the Tegaderm® commercial wound dressing which is a product of the company 3M. The Tegaderm® is a wound dressing that is polyurethane polymer-based, elastic, self-adhesive, hypoallergenic and water-resistant. Said wound dressing has the structure of a transparent film and does not contain any wound healing active substances.

TABLE 2
Average and standard deviation values of the changes
occurring in the wound area

| Sample | Day 1 | Day 3 | Day 5 | Day 7 | Day 9 | Day 21 |
|---|---|---|---|---|---|---|
| Polyurethane | 0 | −2.4 ± | 11.6 ± | 37.6 ± | 60.8 ± | 95.5 ± |
| nanofibrous mat | | 6.6 | 7.1 | 6.8 | 8.2 | 0.9 |
| Polyurethane | 33.1 ± | 37.5 ± | 39.9 ± | 67.3 + | 88.3 ± | 98.4 ± |
| nanofibrous | 3.1 | 4.2 | 3.5 | 2.1 | 1.5 | 0.3 |
| mat containing | | | | | | |
| black seed oil | | | | | | |
| Tegaderm | 8.4 ± | 18.8 ± | 40.4 ± | 56.4 ± | 62.4 ± | 90.6 ± |
| Commercial | 1.4 | 3.9 | 3.7 | 3.8 | 5.9 | 1.6 |
| WoundDressing | | | | | | |

In conclusion, when comparing the wound closure rates obtained on all of the control days of the experiment, the earliest wound closure has been observed on the area where the polyurethane nanofibrous mat containing black seed oil has been utilized. The area where respectively the control group and the polyurethane nanofibrous mat are utilized comes after the first area. The reason that the polyurethane nanofibrous mat containing black seed oil exhibits a better wound healing performance compared to said commercial wound dressing can be evidently associated with; the effect of the black seed oil used as an additive in the nanofibers forming the surface as well as the advantageous properties of the nanofibrous mat through the use of the electrospinning method.

1. Nanofibrous wound dressing comprising black seed oil and polymer.

2. The wound dressing according to claim 1, wherein the polymer is selected from a group consisting of polyurethane polymer, polyacrylonitrile polymer, polyester polymer, polylactic acid, cellulose acetate, poly (e-caprolactone), polyethylene oxide, alginate, polyvinyl alcohol, fibroin, hyaluronic acid, and polycaprolactone.

3. A method for producing a wound dressing according to claim 1, comprising the steps of:
extracting black oil from black seed,
dissolving the polymer in a solvent,
adding the black seed oil into the polymer solution obtained,
obtaining a nanofibrous mat with a method of electrospinning from the solution containing the polymer, solvent and black seed oil.

4. The method for producing a wound dressing according to claim 3, wherein the polymer is selected from a group consisting of polyurethane polymer, polyacrylonitrile polymer, polyester polymer, polylactic acid, cellulose acetate, poly (e-caprolactone), polyethylene oxide, alginate, polyvinyl alcohol, fibroin, hyaluronic acid, and polycaprolactone.

5. Nanofibrous wound dressing according to claim 3, wherein the step of the process of extracting black seed oil from black seed comprises the use of a cold pressing method.

6. The method for producing a wound dressing according to claim 5, wherein the cold pressing method having an operating temperature of a maximum 30-40° C.

7. The method for producing a wound dressing according to claim 3, comprising the step of the process of dissolving 5%-18% polymer (w/v) by weight in a solvent.

8. The method for producing a wound dressing according to claim 3, wherein the step of the process of dissolving the polymer with the solvent comprises mixing a magnetic mixer at the temperature range of 30° C. to 45° C.

9. The method for producing a wound dressing according to claim 3, wherein the step of the process of adding black seed oil is in the range of 5%-20% by volume.

10. The method for producing a wound dressing according to claim 3, comprising the step of the process of applying electrospinning with a feeding rate in the range of 0.5 mL/hour-1.5 mL/hour.

11. The method for producing a wound dressing according to claim 3, comprising the step of the process of applying electrospinning such that the distance between the feeding unit and the collector is 8 cm to 30 cm.

12. The method for producing a wound dressing according to claim 3, comprising the step of the process of applying electrospinning at a voltage at the range of 6 kV-30 kV.
